(19) United States
(12) Patent Application Publication
(10) Pub. No.: US 2025/0205506 A1
(43) Pub. Date: Jun. 26, 2025

(54) Treatment of viral infection

(71) Applicant: John Vivian Cridland, Chippenham (GB)

(72) Inventor: John Vivian Cridland, Chippenham (GB)

(21) Appl. No.: 18/778,987

(22) Filed: Jul. 21, 2024

(30) Foreign Application Priority Data
Dec. 22, 2023 (GB) GB2319984.7

(51) Int. Cl. A61N 5/06; A61K 9/06; A61K 31/573; A61K 31/708; A61P 31/22

(52) U.S. Cl. CPC A61N 5/06; A61K 9/06; A61K 31/573; A61K 31/708; A61P 31/22; A61N 2005/0663

(57) ABSTRACT

The invention relates to the treatment of viral infections in mammals including humans comprising the administration of medicaments or compositions with antiviral properties in combination with illumination with light with wavelength bands that have therapeutic effect or a combination of such wavelength bands that have therapeutic effect. In particular the present invention relates to treatment of viral infection with topical, oral, or intravenous acyclic nucleosides with antiviral properties such as acyclovir, valacyclovir, penciclovir or famciclovir combined with illumination with light that has therapeutic effect such light including wavelength bands in the wavelength range 400 nm to 460 nm.

### DEFINITIONS

As used herein the following definitions apply:

The term “acyclovir” as used herein includes “aciclovir” and vice versa. Acyclovir refers to 9-(2-hydroxyethoxymethyl) guanine as disclosed in U.S. Pat. No. 4,199,574 and Patent EP1750718. It is used as an antiviral which inhibits human herpes viruses, including herpes simplex types 1 (HSV-1) and 2 (HSV-2), varicella zoster, Epstein-Barr virus (EBV) and cytomegalovirus (CMV). The term acyclovir as used herein also includes acycloguanosine.

The term “valacyclovir” as used herein includes “valaciclovir” and vice versa. Valacyclovir is a prodrug of acyclovir. It is used as an antiviral which inhibits human herpes viruses, including herpes simplex types 1 (HSV-1) and 2 (HSV-2), herpes zoster and varicella zoster virus. Valacyclovir and its pharmaceutically acceptable salts are disclosed in U.S. Pat. No. 4,957,924 and WO2017/149420. The term valacyclovir also includes herein its pharmaceutically acceptable salts and valacyclovir hydrochloride an antiviral drug approved in United States and several other countries under the trade name VALTREX®. The term valacyclovir as used herein also includes, ValACV, zelitrex.

The term “penciclovir” as used herein includes “pencyclovir” and vice versa. It is used as an antiviral which inhibits human herpes viruses, including herpes simplex types 1 (HSV-1) and 2 (HSV-2), varicella zoster and Epstein-Barr viruses. Penciclovir herein also includes its salts, phosphate esters and acyl derivatives thereof as antiviral agents as disclosed in patents U.S. Pat. No. 6,124,303, WO91/11187, EP0141927, EP0416739. The term pencilovir as used herein also includes denavir, penciclovirum.

The term “famciclovir” as used herein includes “famcyclovir” and vice versa. Famciclovir is a prodrug of penciclovir. It is used as an antiviral which inhibits human herpes viruses, including herpes simplex types 1 (HSV-1) and 2 (HSV-2), varicella zoster and hepatitis B virus. Famciclovir as disclosed in WO2005/026167 as used herein also includes famciclovir monohydrate as disclosed in WO97/29108. The term famciclovir as used herein also includes famvir, famciclovirum.

The term “steroid” shall mean herein a “corticosteroid”.

Herein the singular shall include the plural and the plural the singular.

### INCORPORATION BY REFERENCE

All publications and patents or patent applications mentioned in this specification are herein incorporated by reference in their entirety to the same extent as if each individual publication or patent or patent application was specifically and individually indicated to be incorporated by reference.

### FIELD OF THE INVENTION

The present invention relates to medicaments, compositions, and phototherapy to treat viral infections. In particular the present invention relates to treatment of viral infections that include but not limited to herpes simplex infections with topical, oral, or intravenous medicaments having antiviral or anti-inflammatory properties or combinations thereof combined with illumination of the affected area with light with wavelength bands or combination of wavelength bands that have therapeutic effect.

### BACKGROUND OF THE INVENTION

The herpes simplex virus (HSV) is a lifelong infection that is present in more than three quarters of the population and may manifest as recurrent herpes labialis (RHL). HSV binds to receptors of the cell membrane, penetrates the cell, and initiates viral replication. Subsequently latent infections are established in sensory nerve ganglia. Recurrent herpes labialis also referred to as “cold sores” is an infectious disease that affect many millions of people worldwide. RHL may be triggered spontaneously or by a wide range of factors e.g., exposure to sunlight or stress etc. Both HSV-1 and HSV-2 virus subtypes affect the skin and mucous membranes, but the orofacial area is most commonly affected by HSV-1. This condition is characterized by a reddened area that may give rise to tingling sensations it may become slightly swollen which progresses to liquid filled vesicles that crusts and eventually heals, the process lasting for approximately 10-14 days. RHL is a self-limiting disease that usually causes painful lesions with some individuals experiencing several outbreaks per year that may have aesthetic impact increasing self-consciousness and limiting social activities. Patients where the immune system has been compromised experience recurrent lesions that are more aggressive and slower to heal.

The treatment of herpes virus infections such as HSV and or herpes zoster virus (HZV) by acyclic nucleosides is well established. Antiviral drugs such as acyclovir (9-(2-hydroxyethoxymethyl) guanine), penciclovir, famciclovir and valaciclovir, in oral or topical form, may be used to treat recurrent herpes labialis. The phosphorylation of these medications or their derivatives and conversion to the triphosphate analog inhibits the synthesis by viral DNA. In particular, acyclovir triphosphate competitively inhibits viral DNA polymerase, incorporates into viral DNA thus stopping DNA-synthesis and terminates virus replication.

Creams, gels, lotions, and ointments containing the drugs acyclovir or penciclovir are available in pharmacies without a prescription. This facilitates the treatment at the earliest sign of symptoms. Treatments with acyclovir or penciclovir are topical creams applied to the affected area a number of times daily. The low bioavailability and half-life of acyclovir requires reapplication at least every 2 to 3 hrs. These over-the-counter antiviral medications can typically reduce the severity of symptoms and shorten cold sore symptoms by a day or two (Spruance S L et al, Antimicrobial. Agents and Chemotherapy, 46, No. 7:2238-2243, (2002) and Van Vloten W A et al, Journal of Antimicrobial Chemotherapy 12, Issue suppl. B: 89-93, (1983)). However, to be effective treatment should commence as early as possible and within 24 hrs of first appearance of symptoms and be applied to the affected area every 2-3 hrs during the waking hours.

The antiviral drugs acyclovir, famciclovir and valacyclovir are also available in the form of tablets but require a prescription. Again, research has shown that these can reduce healing time by about one day in otherwise healthy people. Valacyclovir taken once daily is an effective preventative treatment for herpes reducing frequency and severity of outbreaks. Tablets are well tolerated but people who have kidney failure may require a lower dose.

Light when delivered to the body has been shown to elicit a wide range of therapeutic effects. In particular light can be used as a therapeutic agent to treat skin disorders such as acne, psoriasis, vitiligo, dermatitis etc. These treatments can be in the doctor's office, hospital or at home and may apply the light to the entire body or parts thereof. It is well known that the treatment of acne with light with wavelength in the range 400-450 nm has therapeutic effect which reduce the occurrence of spots and lesions and also speeds up healing. For the treatment of acne there are light based products on the market that are designed to be used at home to provide effective treatment of the condition.

Low Level Light Therapy (LLLT) with wavelengths in the range 630 nm to 890 nm has been used with qualified success in clinical trials and case reports to treat herpes infections (Moskvin S V, J Lasers Med Sci. 12: e38 (2021), Ferreira et al, Rev. Soc. Bras. Med. Trop. 44 (3): 397-399 (2011) and Shindl A & Neumann R, J Invest Dermatol. 113 (2): 221-3 (1999)). In addition, reduced healing times for the treatment of herpes infections with longer wavelength in the NIR have also showed success (Dougal G & Kelly P, Clinical & Experimental Dermatology, 26:149-154 (2001) and Hargate G, Clin. Exp. Dermatol. 31 (5): 638-41 (2006)).

Photodynamic therapy (PDT) has emerged as a therapeutic option in many fields of medicine, such as dermatology and oncology, and is also a promising modality for the treatment of lesions affecting the oral and facial regions. PDT is based on the chemical interaction between a photosensitizer and a light source in a tissue, causing selective cell damage due to the production of reactive oxygen species. Methylene Blue is a well-known sensitiser that has been used to treat recurrent herpes infections (Khalil M & Hamadah O, Photobiomodul. Photomed. Laser Surg. 40 (5): 299-307 (2022) and Lotufo M A et al, Photodiagnosis. Photodyn. Ther. 29:101536, (2020)). The downside of using a photosensitiser such as methylene blue, which is a blue dye, is that it may leave an unsightly stain. In addition, this treatment in some cases requires the vesicle to be drained with a needle before treatment, this requires a medical practitioner, may be painful and risks further infection.

Combination treatment using 5% acyclovir combined with 1% hydrocortisone in a novel cream showed synergistic antiviral activity compared to acyclovir alone when applied to herpes simplex labialis (HSL) (Hull C M et al, J Am Acad. Dermatol. 64 (4): 696, e1-11, (2011)). A cream to treat cold sores that also contains the two active substances acyclovir and hydrocortisone is marketed as Zovirax Duo®. Another combination treatment of HSL with valacyclovir and clobetasol have also shown reduced healing times (Hull C M, Brunton S, Postgrad Med. 122 (5), p 1-6, (2010) abstract).

The beneficial effects of red and NIR light have been described herein. These wavelengths have good transmission through skin and can penetrate many millimetres. The combined effects of orally administered acyclovir and red light from a HeNe laser have been shown to offer improvements over acyclovir alone in the treatment of herpes infections (Velez-Gonzalez et al, -In: Effects of Low-Power Light on Biological Systems. Vol 2630 SPIE; (1996)). Additionally, the combination treatment of intravenously administered acyclovir and the application of golden light (combined 565 nm yellow light and 630 nm red light) or red light to the affected area have shown an improvement over acyclovir alone for treating herpes zoster (Li S et al, Lasers in Medical Science, 38, Article number: 157, (2023)). Administering acyclovir intravenously requires a hospital or outpatient setting, has to be performed under medical supervision and therefore restricts its availability to herpes virus suffers.

### SUMMARY OF THE INVENTION

As used herein the following definition also applies:

The term “therapy light” refers to light with one or more wavelength bands in the wavelength range 400 nm to 460 nm or such wavelength bands in combination with one or more wavelength bands in the wavelength range 465 nm to 2000 nm.

The present invention seeks to improve healing times and severity of viral skin infections, in particular the herpes virus, by the combined administration of an antiviral medicament or combination of medicaments and illumination of the affected area with therapy light where the treatment could be performed at home or in a non-hospital or non-outpatient setting.

A preferred embodiment of the present invention provides a treatment of herpes wherein the herpes-affected area is treated with a topical cream, ointment, lotion, gel or oil that contains an acyclic nucleoside or acyclic guanosine medicament and the said affected and treated area is illuminated with therapy light for treatment thereof and maintain the therapy light radiation for a prescribed treatment duration.

Further in accordance with a preferred embodiment of the present invention there is provided a treatment of herpes wherein the herpes-affected area is treated with a topical cream, ointment, lotion, gel or oil that contains acyclovir as the medicament and the said affected and treated area is illuminated with therapy light for treatment thereof and maintain the therapy light radiation for a prescribed treatment duration.

Still further in accordance with a preferred embodiment of the present invention there is provided a treatment of herpes wherein the herpes-affected area is treated with a topical cream, ointment, lotion gel, or oil that contains penciclovir as the medicament and the said affected and treated area is illuminated with therapy light for treatment thereof and maintain the therapy light radiation for a prescribed treatment duration.

Still further in accordance with a preferred embodiment of the present invention there is provided a treatment of herpes wherein the herpes-affected area is treated with a topical cream, ointment, lotion, gel or oil that contains an acyclic nucleoside or acyclic guanosine medicament combined with a steroid medicament and the said affected and treated area is illuminated with therapy light for treatment thereof and maintain the therapy light radiation for a prescribed treatment duration.

Still further in accordance with a preferred embodiment of the present invention there is provided a treatment of herpes wherein the herpes-affected area is treated with a topical cream, ointment, lotion, gel or oil that contains an acyclovir medicament combined with a hydrocortisone medicament and the said affected and treated area is illuminated with therapy light for treatment thereof and maintain the therapy light radiation for a prescribed treatment duration.

Further for such topical administration the creams, ointment, lotions, gels, or oils aforementioned that contain antiviral medicaments and other medicaments may also include skin penetration enhancers such as dimethylsulphoxide (DMSO), propylene glycol, polyethylene glycol, isopropanol, oleic acid, and the like to be included to increase permeability of the skin. The creams, ointment, lotions, gels or oils may also contain emulsion or suspension stabilisers, surfactants and the like or other pharmaceutically acceptable materials as required.

Still further in accordance with a preferred embodiment of the present invention there is provided a treatment of herpes wherein an acyclic nucleoside or acyclic guanosine medicament is administered either orally or intravenously and the herpes-affected area is illuminated with therapy light for treatment thereof and maintain the therapy light radiation for a prescribed treatment duration.

Still further in accordance with a preferred embodiment of the present invention there is provided a treatment of herpes wherein the medicament is any one or combination of acyclovir, valacyclovir, famciclovir administered either orally or intravenously and the herpes-affected area is illuminated with therapy light for treatment thereof and maintain the therapy light radiation for a prescribed treatment duration.

For oral, topical or parenteral administration the medicament may be combined with a pharmaceutically acceptable carrier in the sense of being compatible with other ingredients of the formulation and not deleterious to the recipient thereof.

Still further in accordance with the preferred embodiment of the present invention the therapy light comprises light with one or more wavelength bands within the wavelength range 400 nm to 430 nm or such wavelength bands in combination with one or more wavelength bands in the wavelength range 465 nm to 2000 nm.

Still further in accordance with the preferred embodiment of the present invention the therapy light comprises light with one or more wavelength bands within the wavelength range 400 nm to 460 nm in combination with one or more wavelength bands in the wavelength range 600 nm to 900 nm.

Further in accordance with a preferred embodiment of the present invention there is provided a treatment for a herpes labialis.

Many substances or materials may be added to the topical, oral or intravenous formulations without affecting the invention as described.

Whilst the action of blue light on treating bacterial skin infection is well known. In particular the absorption of blue light by porphyrin molecules in the bacteria cells leading to generation of free radicals that damage or kill the bacteria it was unexpected to see a beneficial effect with cold sores caused by the herpes simplex virus.

### DETAILED DESCRIPTION

The present invention is described herein using several definitions, as set forth below and throughout the application.

The term “treat” as used herein includes: Relieving or alleviating at least one symptom of an ailment or disease in a mammal. Relieving or alleviating the intensity and or duration of a manifestation of an ailment or disease experienced by a mammal, including prophylactically preventing, curing, healing, easing, remedying, improving, or effecting a condition (e.g., a disease), the symptoms of the condition or the predisposition towards the condition. Delaying the onset of the ailment by for example increasing the period between recurrent incidences of the disorder.

The term “pharmaceutically acceptable” as used herein refers to additives or compositions that are physiologically tolerable and do not substantially produce an allergic, immunological or similar undesirable reaction when administered to a mammal.

The term “Subject” as used herein refers to organisms to be treated by the composition of the present invention. Such organisms include animals and humans.

As used herein the term “topically” refers to application of the compositions of the present invention to the surface of the skin or mucosal cells or tissues.

As used herein the terms ‘healing time’ or ‘time to heal’, is the time from first appearance of herpes labialis symptoms of the recurrence to the disappearance of the crust which may leave some residual redness or erythema.

The term “administered” refers to the means of delivering a medicament to a subject which includes topical, oral, and intravenous methods.

The term “fluence” refers to the energy of therapy light delivered to the subject's tissue per unit area.

### Therapy Light Sources

### Source #1

Source #1 was a battery powered handheld device comprising eight 405 nm LEDs arranged in an array. The wavelength band was 400-410 nm with a full width half maximum (fwhm) of 5.3 nm. The arrangement produced a smooth illumination pattern at a distance 40 mm from the plane of the emitters that had an intensity distribution with a fwhm of 27 mm in the long axis and a fwhm of 16 mm in the short axis. In addition, the beam was reasonably uniform, within 10%, in the central area for ˜10 mm in the short axis and ˜20 mm in the long axis. The unit had a built-in timer which switched the LEDs off 60 seconds after being activated. The intensity or power density of therapy light in the central region of the intensity distribution at a distance of 40 mm from the plane of the emitters was typically 27 mW/cm². Therefore, one exposure from the unit gave a fluence of 1.6 J/cm².

### Source #2

Source #2 was a handheld unit powered by an AC adaptor. This unit had two 420 nm LEDs with wavelength band of 400-440 nm and a fwhm of 14 nm. The arrangement produced a smooth illumination pattern at a distance 15 mm from the plane of the emitters. The illumination pattern had a fwhm of 35 mm in the long axis and a fwhm of 23 mm in the short axis. In addition, the beam was reasonably uniform, within 10%, in the central area for ˜11 mm in the short axis and ˜16 mm in the long axis. This unit also had a built-in timer that switched off the LEDs 60 seconds after being activated. The intensity or power density in the central region of the intensity distribution a distance 15 mm from the plane of the emitters was 70 mW/cm² giving a fluence of 4.2 J/cm² per exposure.

### Source #3

Source #3 was a battery powered handheld device comprising eight 400 nm LEDs arranged in an array. The wavelength band was 395-405 nm with a full width half maximum (fwhm) of 5.0 nm. The illumination pattern at 40 mm from the plane of the emitters was similar to Source #1. This unit also had a built-in timer, but its exposure time was 50s. The intensity or power density of therapy light in the central region of the intensity distribution at a distance of 40 mm from the plane of the emitters was typically 17.1 mW/cm². Therefore, one exposure from the unit gave a fluence of 0.86 J/cm².

All therapy light sources illumination patterns were such that a lesion of less than or equal to 10 mm in size received approximately uniform intensity of therapy light when illuminated by the central area of the intensity distribution of each light source.

The present invention supported in the following Example Section should not be construed to limit in any way the invention as defined in the claims which follow thereafter.

### EXAMPLES

The following examples relate to the acyclic nucleoside acyclovir administered in a topical cream marketed by Boots Ltd called Boots Antiviral Cold Sore (BACS) cream. The person skilled in the art will understand that other antiviral medicaments or combinations thereof or combinations of antiviral medicament and steroid medicament may also be used in combination with illumination with therapy light. Therefore, the examples herein should in no way limit the field of the invention as defined in the claims.

Over a period of 4 years one female, Subject 1, and one male, Subject 2, that suffered from occasional cold sore recurrence were asked to treat recurrences of herpes simplex labialis (HSL) with acyclovir and therapy light. Acyclovir was applied in the form of Boots Antiviral Cold Sore (BACS) cream that contained acyclovir at 5% w/w. The application of BACS cream and therapy light was conducted by the patient themselves in their own home. Subject 1 had access to therapy light Source #1 and tubes of BACS cream which could be used at the onset and during a cold sore recurrence. Subject 2 had access to BACS cream, light Source #1, light Source #2 and light Source #3. Some guidelines were given to both Subjects on how to use the therapy light sources, but it was up to the Subjects to decide the frequency of application and illumination based on lifestyle etc.

The Subjects were instructed to direct the selected therapy light sources at the affected area and position the emitting aperture of Source #1 or Source #3 approximately 40 mm from the treatment area and Source #2 approximately 15 mm from the treatment area. The Subject would then activate the unit maintain the light source in position and wait for the unit's internal timer to switch off the light output. The Subject could then apply another exposure, if so desire d, by turning on the light source and waiting for light emission to terminate. The illumination of the affected area could then be repeated to give one, two or three exposures in a treatment. It should be understood that treatment was conducted in daylight or room light and hence other wavelengths were also incident on the area being treated. These other wavelengths may have a combined effect with the light from Source #1, Source #2, or Source #3.

Photographs of the Subjects face, including the affected area were taken at the start and at intervals during the treatment. The date and time of the photographs were also recorded. The photographs allowed the severity and duration of the recurrence to be determined.

The following examples illustrate the invention without limitation.

### Example 1

Subject 1 aged 72 years that experiences cold sore recurrences about two times per year. At the first sign or symptoms of a cold sore recurrence BACS cream with active ingredient acyclovir at 5% w/w concentration was applied to the affected area. Following the application of BACS cream, the affected area was illuminated with therapy light from Source #1. For day one to day three of treatment BACS cream was applied approximately nine or ten times per day during waking hours the number of applications decreases steadily to one application at day eight of treatment. Each application was typically followed by one exposure of therapy light from Source #1. The subject's face including the affected area was photographed at the beginning and during the treatment. The subject reported that the severity and size of the blister or vesicle was reduced as was the pain compared to previous recurrences that were only treated with BACS cream. The size of the largest dimension of the crust was seen from photographs to be ˜1 mm. The vesicle had subsided by day four of treatment, crust formed between days five to seven and healing of crust at day eight of treatment.

Exposure to therapy light was typically applied one to five minutes after BACS cream application but not exclusively. On some occasions illumination occurred sometime between one application of BACS cream and the next.

### Example 2

The same Subject 1 aged 72 yrs reported in Example 1 with a cold sore recurrence after a further six months. At the first sign of symptoms of the cold sore recurrence BACS cream with 5% w/w acyclovir was applied to the affected area and within a period of one to five minutes, following the application of BACS cream, the treated area was illuminated with therapy light from Source #1. For the first three days of treatment BACS cream was applied approximately six times daily, during waking hours. Each application of BACS cream was followed by two or three therapy light exposures of 60s. The number of applications of BACS cream and exposure to therapy light decreased steadily from day four of treatment with only one application and exposure at day nine of treatment. The corresponding fluence per day was 24 J/cm² for the first two days of treatment dropping to 2 J/cm²/day at end of treatment.

The region of the face including the affected area was photographed at the beginning and during the treatment. The subject again reported that the pain, the severity, and size of the vesicle was reduced compared to previous recurrences that were only treated with BACS cream. The size of the largest dimension of the crust was seen from photographs to be ˜1 mm. The vesicle had subsided by day three of treatment, crust formed between days four and five and healing of crust at day five of treatment.

### Example 3

Subject 2 aged 65 yrs that experiences occasional (<1 per year) cold sore recurrences. The subject reported regular application of BACS cream and illumination with therapy light from Source #2, with six applications in the first day of treatment dropping to one application by day five of treatment. Typically, the affected area was illuminated with one exposure of therapy light source #2 between two and thirty minutes after BACS cream application.

The face including the affected area was photographed. The blister phase had subsided by day three of treatment with the crust, which was approximately 4 mm in size, also beginning to form on day three of treatment. The crust had gone between days four and six of treatment (mean value-day five) leaving a slightly red area.

### Example 4

The same Subject 2 aged 67 yrs. The subject reported three application of BACS cream with one exposure of therapy light from Source #1 following each application of BACS cream for the first two days of the recurrence. The application of BACS cream and illumination were increased to six or seven applications per day for days three and four of treatment decreasing to four applications per day for days five, six & seven and one application at day eight of treatment. The application of therapy light from Source #1 gave a fluence of 2.6 J/cm²/day for the first two days of treatment increasing to 3 to 5 J/cm²/day for days three to six of treatment. However, days three & four of treatment were also subject to additional therapy light exposure from Source #2 at a fluence of 12 and 16 J/cm²/day respectively.

The face including the affected area was photographed at the start and during treatment. The blister or crust was approximately 3 mm in size. The blister phase had subsided by day four of treatment and crust beginning to form on day five of treatment. The crust had gone by day eight of treatment leaving a dry and red region that persisted till day thirteen.

### Example 5

The same Subject 2 aged 69 yrs. At the first sign or symptoms of the cold sore recurrence, BACS cream was applied to the affected area, then within one to fifteen minutes the area was illuminated with therapy light from Source #1 or from Source #2. The area was also illuminated at other times between applications of BACS cream. There were eight application of BACS cream during day one of treatment followed by three or four applications per day for days two to six of treatment dropping to one application at day nine of treatment. The illumination with therapy light from Source #2 was relatively high on the first day of treatment at ˜29 J/cm²/day dropping to 20 J/cm²/day for days two and three of treatment and decreasing thereafter. Exposure to therapy light from Source #1 was ˜11 J/cm²/day for the first day of treatment dropping to <4 J/cm²/day between days two to seven of treatment.

The face including the affected area was photographed. The blister was approximately 3 mm in size and this phase had subsided by day two of treatment and crust also beginning to form on day two of treatment. The crust had gone by day six of treatment leaving a small red region.

It should be noted that BACS cream was continued to be applied and illuminated for a few days after healing time for all examples.

Table 1 presents the healing time, size of crust or blister, number of BACS cream applications for the first day and the fluence of therapy light administered to the herpes-affected area during the first day for examples 1 to 5 herein.

TABLE 1

| | | | Mean | 1^{st} day | 1^{st} day |
|---|---|---|---|---|---|
| | | Time | crust/ | No. of | therapy light |
| | | to heal | blister | BACS cream | fluence |
| Example | Subject | (days) | size (mm) | applications | (J/cm²) |
| 1 | 1 | 8 | 1.1 | 10 | 16 |
| 2 | 1 | 5 | 1.2 | 6 | 24 |
| 3 | 2 | 5 | 4.0 | 6 | 25.2 |
| 4 | 2 | 8 | 3.0 | 3 | 2.6 |
| 5 | 2 | 6 | 3.3 | 8 | 39.8 |

With reference to table 2 which presents the healing time listed against the fluence of therapy light administered, from Source #1, Source #2, and Source #3 to the herpes-affected area on day one of treatment for each of the five examples. Table 2 shows a decrease in the mean healing time from 8 days for therapy light fluences <20 J/cm² to a mean value of 5.33 days for therapy light fluence >20 J/cm². Similar decreasing trends in healing time were observed with respect to the combined therapy light fluence for the first two days of treatment or with respect to the total therapy light fluence administered up to and including the healing time.

TABLE 2

| 1^{st} day | |
|---|---|
| therapy light | |
| fluence | Time to |
| (J/cm²) | heal (days) |
| 2.6 | 8 |
| 16 | 8 |
| 24 | 5 |
| 25.2 | 5 |
| 39.8 | 6 |

Referring now to table 3 the healing time when listed against the number of BACS cream applications does not show the same decrease in healing time with increased BACS cream applications. It must be remembered that each BACS application had a corresponding therapy light illumination. However, the fluence of therapy light was not consistent for each BACS cream application. In particular, the number of exposures varied between one and three and the power density and fluence per exposure varied depending on the light source. This means there was not a fixed correlation between BACS cream applications and therapy light fluence. Therefore, the fact that table 2 shows a decrease in healing time with therapy light fluence >20 J/cm² for the first day of treatment and table 3 does not have a corresponding correlation, demonstrates that the combination of BACS cream and therapy light of sufficient fluence shows a synergistic antiviral effect particularly with regard to healing time.

TABLE 3

| 1^{st} day | |
|---|---|
| No. of BACS cream | Time to |
| applications | heal (days) |
| 3 | 8 |
| 6 | 5 |
| 6 | 5 |
| 8 | 6 |
| 10 | 8 |

The female subject 1 reported that the main benefit of BACS cream and therapy light was the reduced severity of the recurrence with much smaller crusts that could be covered more easily with makeup together with reduced healing time when compared to treatment with BACS cream only.

It will be appreciated by persons skilled in the art that the present invention is not limited to what has been particularly shown and described hereinabove. Rather the scope of the present invention is defined only by the claims that follow.

1. A composition comprising an antiviral medicament or combination of antiviral and other medicaments for use in the treatment of a viral infection by combined, sequential or separate administration with illumination of the area to be treated by light comprising one or more wavelength bands within the wavelength range 400 nm to 460 nm or such wavelength bands in combination with one or more wavelength bands in the wavelength range 465 nm to 2000 nm.

2. A composition for use according to claim 1 wherein the viral infection is herpes virus.

3. A composition for use according to claim 2 wherein the antiviral medicament or combination of antiviral and other medicaments includes one or more acyclic nucleoside.

4. A composition for use according to claim 3 wherein the acyclic nucleoside is any one or combination of acyclovir, penciclovir, valacyclovir or famciclovir.

5. A composition for use according to claim 4 wherein any one or combination or all of acyclovir, penciclovir, valacyclovir or famciclovir is in the concentration range 0.01 to 20% w/w or dose in the range 5 mg to 5000 mg.

6. A composition for use according to claim 4 wherein acyclovir is in the concentration range 0.01 to 20% w/w or dose in the range 5 mg to 5000 mg.

7. A composition for use according to claim 2 wherein the antiviral medicament or combination of antiviral and other medicaments includes one or more acyclic nucleoside medicament and one ((of)) or more steroid.

8. A composition for use according to claim 7 wherein the acyclic nucleoside is any one or combination of acyclovir, penciclovir, valacyclovir, famciclovir.

9. A composition for use according to claim 8 wherein the steroid is any one or combination of hydrocortisone and clobetasol.

10. A composition for use according to claim 9 wherein the acyclic nucleoside is acyclovir and the steroid is hydrocortisone.

11. A composition for use according to claim 8 wherein any one or combination or all of acyclovir, penciclovir, valacyclovir or famciclovir is in the concentration range 0.01 to 20% w/w or dose in the range 5 mg to 5000 mg.

12. A composition for use according to claim 10 wherein either or both acyclovir or hydrocortisone is in the concentration range 0.01 to 20% w/w or dose in the range 5 mg to 5000 mg.

13. A composition for use according to claim 5 wherein the medicaments are administered in the form of creams, lotions, ointments, oils, tablets, capsules, liquids, suspensions, or emulsions.

14. A composition for use according to claim 9 wherein the medicaments are administered in the form of creams, lotions, ointments, oils, tablets, capsules, liquids, suspensions, or emulsions.

15. A composition for use according to claim 5 wherein the light incident on the treatment area has one or more wavelength bands in the wavelength range 400 nm to 440 nm or such wavelength bands in combination with one or more wavelength bands in the wavelength range 465 nm to 2000 nm.

16. A composition for use according to claim 9 wherein the light incident on the treatment area has one or more wavelength bands in the wavelength range 400 nm to 440 nm or such wavelength bands in combination with one or more wavelength bands in the wavelength range 465 nm to 2000 nm.

17. A composition for use according to claim 15 wherein the light incident on the treatment area has one or more wavelength bands in the wavelength range 400 nm to 440 nm in combination with one or more wavelength bands in the wavelength range 500 nm to 900 nm.

18. A composition for use according to claim 16 wherein the light incident on the treatment area has one or more wavelength bands in the wavelength range 400 nm to 440 nm in combination with one or more wavelength bands in the wavelength range 500 nm to 900 nm.

19. A composition for use according to claim 15 wherein the light incident on the treatment area with one or more wavelength bands within the wavelength range 400 to 440 nm has power density of 0.1 to 2000 mW/cm².

20. A composition for use according to claim 16 wherein the light incident on the treatment area with one or more wavelength bands within the wavelength range 400 to 440 nm has power density of 0.1 to 2000 mW/cm².

21. A method for treating herpes ((a)) viral infection comprising the administration of one or more antiviral medicaments or an antiviral combined with another medicament and combined, sequential or separate illumination of the area to be treated by light comprising one or more wavelength bands within the wavelength range 400 nm to 460 nm or such wavelength bands in combination with one or more wavelength bands in the wavelength range 465 nm to 2000 nm.

22. A method for use according to claim 21 wherein the antiviral medicament is any one or combination of acyclovir, penciclovir, valacyclovir, famciclovir, and wherein the other medicaments is any one or combination of hydrocortisone or clobetasol.
